(19) United States
(12) Patent Application Publication
(10) Pub. No.: US 2026/0301946 A1
(43) Pub. Date: Oct. 1, 2026

(54) ACCURATE LEARNING MODELS FOR TISSUE CHARACTERIZATION

(71) Applicant: IR MEDTEK LLC, Gahanna, OH (US)

(72) Inventors: Rebecca C. BRADLEY, Columbus, OH (US); Douglas J. COHEN, Bexley, OH (US)

(21) Appl. No.: 19/474,026

(22) PCT Filed: Apr. 12, 2024
(86) PCT No.: PCT/US2024/024190

§ 371 Date: Oct. 9, 2025

Related U.S. Application Data
(60) Provisional application No. 63/495,685, filed on Apr. 12, 2023.

(51) Int. Cl. G16H 50/20; A61B 5/00; G16H 10/40; G16H 10/60; G16H 15/00; G16H 30/20

(52) U.S. Cl. CPC G16H 50/20; A61B 5/7267; G16H 10/40; G16H 10/60; G16H 15/00; G16H 30/20

(57) ABSTRACT

Systems and methods can employ optical spectroscopic tissue characterization such as for cancer detection or diagnosis, such as to training and using accurate learning models for tissue characterization. The present approach can include one or more of: (1) using the preliminary screening test before proceeding to actual tissue characterization or classification; (2) selecting between different specific trained learning models, such as based on information from the preliminary screening test, user input information, or other information; and (3) presenting information to a user about the tissue characterization or classification, such as which can convey a binary tissue characterization or classification and a degree of confidence or similarity to one or both classes in the binary tissue characterization or classification.

### CLAIM OF PRIORITY

This application claims the benefit of priority of U.S. Provisional Patent Application Ser. No. 63/495,685, entitled TRAINING AND USING ACCURATE LEARNING MODELS FOR TISSUE CHARACTERIZATION, filed Apr. 12, 2023 (Attorney Docket No. 5829.001PRV), which is hereby incorporated herein by reference in its entirety.

### TECHNICAL FIELD

This document pertains generally, but not by way of limitation, to infrared optical spectroscopic tissue characterization such as for cancer detection or diagnosis, and more particularly, but not by way of limitation, to training and using accurate learning models for tissue characterization.

### BACKGROUND

Coe et al. U.S. Patent Application Publication No. US 2019/0110687 A1 entitled SYSTEM AND METHOD FOR THE DISCRIMINATION OF TISSUES USING A FAST INFRARED CANCER PROBE, which is hereby incorporated herein by reference, and which published on Apr. 18, 2019, relates to using an infrared probe and discriminating software to rapidly discriminate normal non-cancerous tissue from abnormal cancerous tissue.

### SUMMARY/OVER VIEW

Infrared (IR) illumination of a tissue sample can be used to perform spectroscopic analysis of the tissue sample, such as to characterize tissue, such as for detecting or diagnosing cancer or another tissue characteristic. A light source, such as one or more tunable quantum cascade laser (QCLs) or other lasers, can be used to perform the IR illumination of the tissue sample. Illumination light can be incoupled into the tissue sample, and response light can be outcoupled from the tissue sample, such as via a crystal tip or a Fiber Loop Probe that can be placed against the tissue sample. Tuning one or more QCLs to scan various illumination wavelengths or wavenumbers, a probe response signal from the tissue sample can be detected, acquired, and analyzed, such as to determine one or more characteristics of the tissue sample.

The present inventors have recognized that, among other things, spectroscopic tissue characterization can face challenges that can impede its accuracy and speed. Both accuracy and speed can be important in a wide-variety of use cases, including characterizing both ex vivo and in vivo tissue samples. For example, both accuracy and speed can be important to a surgeon seeking to use a spectroscopic tissue characterization tool in real-time (or near real-time) such as to determine surgical margin for excising abnormal (e.g., cancerous) tissue vs. normal (e.g., healthy or non-cancerous) tissue. However, certain confounding factors can impede one or both of accuracy and speed of spectroscopic tissue characterization.

First, different body locations may have different spectral characteristics. For example, an external location on a subject's hand may have different spectroscopic tissue characteristics than an external skin location the subject's face. Further, the location on the subject's face may have different spectroscopic tissue characteristics depending on how much facial hair is present, which may be different for a male subject than for a female subject. The composition of hair and fingernails includes keratin, which may have different spectroscopic tissue characteristics than less keratinous tissue. Similarly, a location within the subject's oral cavity (e.g., on the subject's tongue or gums) may have different spectroscopic tissue characteristics than an external skin location. Still further, an internal tissue location (e.g., within a subject's breast) may have different spectroscopic tissue characteristics than an external tissue location (e.g., at an external location on the same breast of the same subject).

Second, a target location on the body to be analyzed may be contaminated, such as by a foreign substance such as one or more of dirt, sunscreen, skin lotion, other cosmetic, rubbing alcohol, soap residue, water, or other cleaning agent, or other contaminant. The molecular composition of the contaminant or contaminants that are present on the skin at the target location to be spectroscopically analyzed and characterized may influence the spectral response obtained at the contaminated target location, which may confound the desired spectroscopic tissue characterization, such as in discrimination between cancerous and non-cancerous tissue.

Third, different specific types of cancers may have different spectroscopic tissue characterization response characteristics. For example, melanoma may present a different spectroscopic response than either or both of squamous cell carcinoma (SCC) or basal cell carcinoma (BCC). Further, the different specific types of cancer may present different tissue characterization spectral response characteristics at different body locations, as similarly described more generally above.

The present inventors have recognized that, while it may be possible to train a learning model that can perform spectroscopic tissue characterization in spite of one or more such variations, confounding factors, or the like, it also may be possible to improve accuracy, speed, or both by making available more specific learning models. Such more specific learning models can be trained for one or a combination of a particular body location, a particular specific cancer type, a particular contaminant, etc. A more specific model can then be selected, such as based on input information indicating whether such a more specific model should be applied. The more specific model can potentially yield better sensitivity, specificity, or both, as compared to a more general model. Such information can be based on user input, patient records, imaging, or a spectroscopic pre-screening test that can be performed before actually beginning the spectroscopic tissue characterization test. The information can be used to select the most appropriate model from a variety of different available models.

The information can additionally or alternatively be used to determine whether to proceed with the actual spectroscopic tissue characterization test. For example, if a preliminary screening test (also referred to as “pre-screening”) indicates that the target external skin location is contaminated with sunscreen or a cosmetic, the user may be instructed to clean the target external skin location. Then, the preliminary screening test can be repeated to determine whether the target external skin location is clean enough to proceed with the actual spectroscopic tissue characterization test. Because the cleaning itself may introduce a different contaminant (e.g., rubbing alcohol, soap, water, or the like), if such different contaminant is determined to be present, e.g., after cleaning, the user may be instructed to further prepare the target external skin location before proceeding beyond pre-screening by commencing with the actual spectroscopic tissue characterization test. Additional information may also be used to select a particular trained tissue characterization model for performing the spectroscopic tissue characterization test. Similarly, additional information may also be used to train such more specific tissue characterization models, such as during a training phase of using the present system for model generation.

This Summary/Overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic diagram illustrating generally an example of portions of a system, such as can be used for characterizing or classifying a tissue specimen.

FIG. 2 is a flow chart illustrating generally an example of portions of a technique that can include a method of training and using one or more learning models for tissue characterization.

FIG. 3 is a spectral response graph of light absorbance vs. wavenumber.

FIG. 4A is a block diagram illustrating an approach of: (1) using the preliminary screening test before proceeding to actual tissue characterization or classification; (2) selecting between different specific trained learning models, such as based on information from the preliminary screening test, user input information, or other information; and (3) presenting information to a user about the tissue characterization or classification, such as which can convey a binary tissue characterization or classification and a degree of confidence or similarity to one or both classes in the binary tissue characterization or classification.

FIG. 4B is a block diagram, similar to that of FIG. 4A, but in which the models can include a condition vs. absence of that condition, rather than a condition vs. “normal.”

FIG. 5 illustrates an example of an evaluation or decision sequence that can be used to proceed towards selecting and applying various specific trained learning models for tissue characterization or classification.

### DETAILED DESCRIPTION

This document pertains generally, but not by way of limitation, to infrared optical spectroscopic tissue characterization such as for cancer detection or diagnosis, and more particularly, but not by way of limitation, to training and using accurate learning models for tissue characterization. This can include obtaining information that can be used to select an appropriately trained learning model from a plurality of different available trained learning models for performing tissue characterization, such as to select a more specific model for use in performing the tissue characterization. This can also include performing one or more preliminary screening tests (“pre-screening”), comparing a result of the preliminary screening test to at least one criterion to determine whether to proceed with the actual tissue illumination and response acquisition for performing the actual tissue characterization, or whether to require another preliminary screening test or some other action (e.g., cleaning a skin location) by the user before proceeding. This document also describes techniques of presenting information to the user that can reflect a determined tissue characterization and an indication of a level of confidence corresponding to the determined tissue characterization.

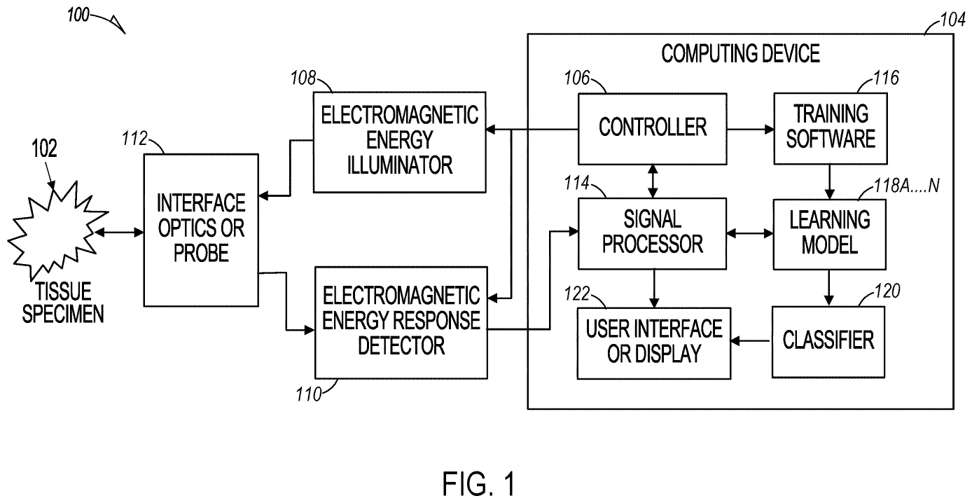

FIG. 1 is a schematic diagram illustrating generally an example of portions of a system 100, such as can be used for characterizing or classifying a tissue specimen 102. The system 100 can include a computing device 104. The computing device 104 can include or be coupled to a controller 106 circuitry. The controller 106 can be configured to provide one or more control output signals, such as to control an electromagnetic energy illuminator 108 and an electromagnetic energy response detector 110, which can be separate components or can be integrated together in a shared component. The illuminator 108 and the response detector 110 can interface with the tissue specimen 102 via one or more of a fiber optic cable or other interface optics or probe component 112. For example, the response detector 110 can include a photodetector or a Focal Plane Array (FPA) imaging array of pixels. Either of these can transduce the response signal into a suitable electrical signal that can be digitized and stored in memory or buffer circuitry and provided to a signal processor 114, circuitry of which can be included in or coupled to the computing device 104. As explained herein, the computing device 104 can include training software 116, such as can be used to train one or more learning models 118A . . . 118N such as which can be stored in memory circuitry included in or communicatively coupled to the computing device 104. The learning models 118 can be included in or coupled to a classifier 120, such as can be used to characterize or classify one or more locations on the tissue specimen 102 into two or more (or three or more, as described herein) different categories. The tissue characterization or classification by the classifier 120 can be displayed or otherwise presented to a diagnostician, clinician, or other user, such as via a local or remote user interface or display 122, such as can be included in or coupled to the computing device 104. In an example, the classification information can be displayed on an enhanced image of portions of the tissue specimen 102, such as can be acquired via the response detector 110 or another imaging device such as can be arranged to gather one or more images from the tissue specimen 102.

The illuminator 108 can be configured for including or using interface optics or a probe 112, such as which can include an Attenuated Total Reflection (ATR) probe, using a Fourier Transform IR Attenuated Total Probe (FTIR-ATR, or “FTIR”) probe, using one or more tunable or fixed-wavelength Quantum Cascade Lasers (QCLs), using an etalon, or using a broadband light source configured to permit spectral filtering or other spectral control of illumination, or other illumination technique, such as described herein. The response detector 110 can be configured for acquiring, detecting, or imaging one or more of a reflection, scattering, fluorescence, Raman, or other electromagnetic energy response signal from the tissue sample or specimen 102. The response detector 110 can be configured for including or using a photodetector, using a Focal Plane Array (FPA) imaging array detector, using the ATR or FTIR probe, using diffuse reflectance IR, or other acquisition technique. Such techniques can be used to generate a spectral response characteristic of the tissue sample or specimen at a particular wavelength or wavelength bin, or such as for generating a spectral response signature of the tissue sample or specimen 102 over multiple illumination wavelengths.

### A. Acquisition of Training Set and Training Learning Model (Overview)

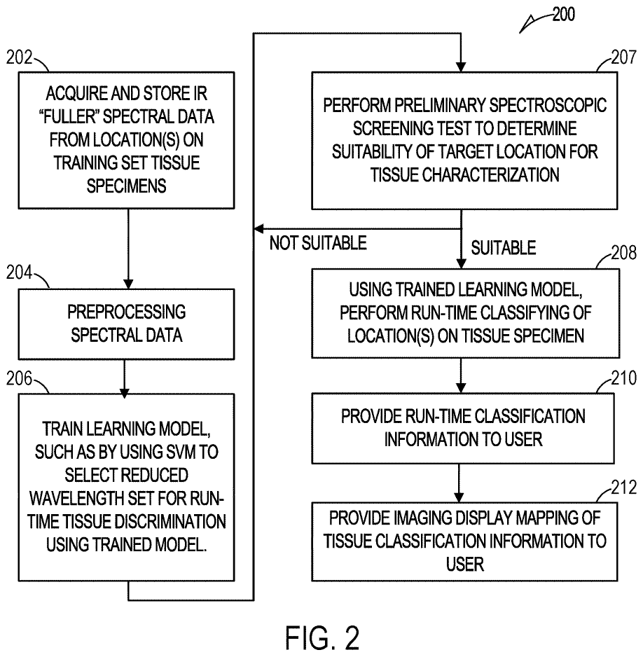

FIG. 2 is a flow chart illustrating generally an example of portions of a technique that can include a method 200 of training and using one or more learning models for tissue characterization, such as the learning model 118. This can include using training software 116 on a computing device 104 to direct the controller 106 for operating the illuminator 108 and the response detector 110 for acquiring mid-range IR spectral data from a training set of tissue specimens 102. For example, the training set of tissue specimens can include in vivo or resected tissue samples from one or more or a population or sub-population of human or animal subjects, appropriately preserved, such as by freezing the tissue samples or the like.

At 202, relatively “fuller” IR spectral data can be acquired from one or more locations on one or more tissue specimens of the training set, and the acquired spectral data can be stored, such as in a memory or database, such as for training the learning model 118. For training the learning model 118, “fuller” wavelength set (e.g., higher spectral resolution spectral data) can be acquired and used for training the learning model 118, as opposed to a “reduced” wavelength set usable by the trained learning model 118 at run-time for quickly and accurately performing multidimensional or other tissue characterization or classification into two or more, three or more, or many multidimensional tissue characterization or classification categories, such as described herein, and such as further described in Coe Jr. et al. U.S. patent application Ser. No. 17/658,489, which is hereby incorporated by reference in its entirety, including for its description of tissue characterization or classification.

As an illustrative example, a practical probe can be used with an FTIR system having an illuminator 108 and response detector 110 and may have a resolution of 4 cm⁻¹ over a mid-IR wavelength range from 900 cm⁻¹ to 1800 cm⁻¹, yielding a response at 450 different wavenumber values (2 cm⁻¹ steps) for use in training the learning model 118. When appropriately trained, such as can include using the techniques described herein, it may be possible to greatly reduce the number of wavenumbers used to classify tissue specimens at run-time using the trained learning model 118, such as by using 10 or fewer selected wavelengths (e.g., ~5 wavelengths) for operating the illuminator 108 and as the response detector 110 at run-time for multi-dimensional or other tissue characterization or classification. The mid-IR spectral range used for training may include certain spectral bands of particular interest for tissue discrimination, such as spectral bands corresponding to the amide I, amide II, amide III, and amide IV bands of protein, or the like, such as described at least in part in Coe et al. U.S. Patent Application Publication No. US 2019/0110687, which is incorporated herein by reference in its entirety.

As another illustrative example, a single or multiple tunable or fixed-wavelength QCL can be used as the illuminator 108 with an appropriate photodetector element or photodetector array of pixels used as the response detector 110. For example, a practical 4 QCL system can provide IR illumination wavelengths between 820 cm⁻¹ and 1680 cm⁻¹, such as with available incremental wavenumber steps being similar to those from an FTIR probe, for example. In another practical example 32 QCLs can be integrated onto a single QCL chip, with an individual one of the 32 QCLs having a 160 cm⁻¹ range tunable in 5 cm⁻¹ steps, with the individual QCL ranges being specifiable to be overlapping, non-overlapping, or partially overlapping to obtain the desired IR spectral range and, if desired, to permit some redundancy in allowing different QCLs to be tuned to the same wavelength. Fixed wavelength lasers can be used instead of (or in addition to) using one or more tunable lasers. Again, for the fixed or tunable QCL approach, the full spectral resolution or a specified subset thereof can be used for training the learning model 118. By appropriate training, the trained learning model 118 can then employ a relatively reduced set of wavelengths at run-time for tissue characterization or classification. By appropriate training, this reduced set of wavelengths used at run-time for tissue characterization or classification using the trained model 118 can optionally be selected to fall within the output range of a single tunable QCL, such that only a single tunable QCL need be tuned and used at run-time for performing the tissue characterization or classification using the trained model 118, if desired.

At 204, regardless of (or depending upon) which technique is to be used for analyzing the spectral data acquired from the response detector 110, it can be desirable to first apply one or more pre-processing techniques to the acquired spectral data. Examples of such spectral techniques can include baseline adjustment, filtering, or the like. One particularly useful pre-processing technique can include applying a second derivative to the acquired spectral data, such as using a specified spectral resolution of applying the second derivative (e.g., skipping a specified number of wavenumbers), such as to help reduce, attenuate, or eliminate an effect of gaseous water vapor otherwise present in the spectral data. An illustrative example and more detailed explanation of applying such a second derivative technique is described in more detail in Coe Jr. et al. U.S. patent application Ser. No. 17/658,489, which is hereby incorporated by reference in its entirety, including for its description of such a second derivative technique.

It is possible to develop one or more metrics for use in analyzing the spectral response data from the response detector 110 for training the learning model 118. For example, such metrics can include one or more of ratios of peak absorbances, calibrant dot product scores, tissue scattering metrics, baseline correction metrics, or the like.

At 206, the pre-processed spectral data from the training data set can be used to train the learning model 118, such as by using artificial intelligence or machine learning techniques for training the learning model 118, such as to obtain a useful reduced set of wavelengths for use at runtime for classifying tissue into tumor and non-tumor categories-as well as into one or more of a variety of other useful diagnostic categories, such as described herein. One example of such a machine learning technique can be referred to as a Support Vector Machines (SVM) learning technique, which can be used to select a reduced wavelength set for run-time tissue discrimination using the trained model 118. As explained in more detail herein, the overall learning model 118 can include a set of multiple specific learning models 118A . . . N, one or more of which can be selected or applied at run-time. For example, information obtained at run-time can be used to select or apply an appropriate learning model 118 from the set of multiple specific learning models 118A . . . N. As also explained in more detail herein, the overall learning model 118 can include at least one learning model 118 that can be used to perform a preliminary screening or pre-screening threshold test for determining whether to proceed with further illumination and response evaluation for tissue characterization and classification.

At 206, the training can include using machine learning support-vector machines (SVMs) which are supervised programs that can use a training data set to obtain an optimized separating surface between different groups such as for tissue characterization or classification. While these different tissue characterization or classification groups can be limited to two groups (e.g., tumor tissue classification group and non-tumor-tissue classification group), the present techniques can also be adapted to classification according to more than two-groups, such as in a complex multi-dimensional space. As explained herein, SVM can be used to develop decision equation values representing hyperdimensional distances from the optimized separating surface. SVMs can provide robust prediction methods. After sufficient training of the learning model 118, resulting decision equations of the trained learning model 118 can be used at run-time to test new tissue specimen spectral data, such as in a clinical or other diagnostic setting. Linear or non-linear SVM techniques, such as using radial basis functions, or other artificial intelligence or machine learning techniques can be used.

At 207, the trained learning model 118 can be used to perform a preliminary screening test at a tissue specimen 102 at an external or internal target location of interest of a patient. The preliminary screening test at 207 can include a mid-range IR pre-characterization illumination and spectroscopic response analysis of the tissue specimen 102 at the target location of interest of the patient, such as using the computing device 104. The preliminary screening test can be used to pre-screen and determine a suitability of the target location of interest for using a trained learning model for performing spectroscopic tissue characterization at the target location. The preliminary screening test can employ a particular trained learning model, e.g., a “suitability” trained learning model 118A that has been trained for making such a preliminary determination of suitability of the target location. Then, a different particular trained learning model, e.g., 118B can be employed, such as with further run-time illumination and response measurements, to actually perform the tissue characterization of the tissue specimen 102 at the target location when the preliminary screening test indicates that the target location is suitable for such further tissue characterization. Otherwise, process flow can return to 207 to perform another preliminary screening test at the same or a different target location to determine suitability. As explained in more detail below, information from the preliminary screening test at 207 (or other information, such as received via the user interface or otherwise) can be used to select a particular trained learning model 118B . . . 118N for use when actually performing the tissue characterization of the tissue specimen 102 at a target location determined suitable by the preliminary screening test at 207.

At 208, the trained learning model 118 can be used at run-time to perform tissue characterization or classification of a tissue specimen 102 at the target location, such as in a diagnostic or clinical setting, such as during a diagnostic or treatment procedure being performed on a patient, such as described herein.

At 210, one or more indications of a result of the run-time tissue characterization or classification can be presented or provided to a diagnostician, clinician, or other user, such as described herein.

At 212, the one or more indications of the result of tissue characterization or classification can optionally be provided as a color (e.g., RGB) or other displayed image provided to the user, such as by mapping at least two—or more than two—tissue classifications to a color coding schema to be provided on the display to the user, such as described herein.

### B. Preliminary Screening Test to Determine Suitability of Target Location

As explained, it may be helpful to perform a preliminary screening test, such as to help determine a suitability of a target location for performing tissue characterization or classification. This is because certain confounding factors can impede one or both of accuracy and speed of spectroscopic tissue characterization.

First, different body locations may have different spectral characteristics. For example, an external location on a subject's hand may have different spectroscopic tissue characteristics than an external skin location the subject's face. Further, the location on the subject's face may have different spectroscopic tissue characteristics depending on how much facial hair is present, which may be different for a male subject than for a female subject. The composition of hair and fingernails includes keratin, which may have different spectroscopic tissue characteristics than less keratinous tissue. Similarly, a location within the subject's oral cavity (e.g., on the subject's tongue or gums) may have different spectroscopic tissue characteristics than an external skin location. Still further, an internal tissue location (e.g., within a subject's breast) may have different spectroscopic tissue characteristics than an external tissue location (e.g., at an external location on the same breast of the same subject).

Second, target location on the body to be analyzed may be contaminated, such as by a foreign substance such as one or more of dirt, sunscreen, skin lotion, other cosmetic, rubbing alcohol, soap residue, water, or other cleaning agent, or other contaminant. The molecular composition of the contaminant or contaminants that are present on the skin at the target location to be spectroscopically analyzed and characterized may influence the spectral response obtained at the contaminated target location, which may confound the desired spectroscopic tissue characterization, such as discrimination between cancerous and non-cancerous tissue.

Third, different specific types of cancers may have different spectroscopic tissue characterization response characteristics. For example, melanoma may present a different spectroscopic response than either or both of squamous cell carcinoma (SCC) or basal cell carcinoma (BCC). Further, the different specific types of cancer may present different tissue characterization spectral response characteristics at different body locations, as similarly described more generally above.

The present inventors have recognized that, while it may be possible to train a learning model that can perform spectroscopic tissue characterization in spite of one or more such variations, confounding factors, or the like, it also may be possible to improve accuracy, speed, or both by making available more specific models. Such more specific models can be trained for one or a combination of a particular body location, a particular specific cancer type, a particular contaminant, etc. A more specific model can be selected based on information indicating whether such a more specific model should be applied. The more specific model can potentially yield better sensitivity, specificity, or both, as compared to a more general model. Such information can be based on user input, patient records, imaging, or a spectroscopic screening test that can be performed before actually beginning the spectroscopic tissue characterization test. The information can be used to select the most appropriate model from a variety of different available models.

The information can additionally or alternatively be used to determine whether to proceed with the actual spectroscopic tissue characterization test. For example, if a preliminary screening test indicates that the target external skin location is contaminated with sunscreen or a cosmetic, the user may be instructed to clean the target external skin location. Then, the preliminary screening test can be repeated to determine whether the target external skin location is clean enough to proceed with the actual spectroscopic tissue characterization test. Because the cleaning itself may introduce a different contaminant (e.g., rubbing alcohol, soap, water, saline, cotton or other fabric residue, or the like), if such different contaminant is determined to be present, the user may be instructed to further prepare the target external skin location before commencing with the actual spectroscopic tissue characterization test. Additional information may also be used to select a particular trained tissue characterization model for performing the spectroscopic tissue characterization test. Similarly, additional information may also be used to train such more specific tissue characterization models, such as during a training phase of using the present system for model generation.

Thus, one or more preliminary screening tests can be performed to help determine a suitability of a target location for performing tissue characterization or classification. Stated differently, a preliminary screening test can include a quick scan of electromagnetic energy illumination at different illumination wavelengths by the electromagnetic energy illuminator 108. The electromagnetic energy response detector 110 can be used to detect corresponding spectral responses to such illuminations. A screening algorithm or a screening trained learning model 118A can be used to determine whether the response spectrum obtained during the preliminary screening test is “tissue-like” or “uncontaminated” enough to apply another trained learning model 118B for actually performing the tissue characterization or classification, such as to characterize the tissue specimen 102 at the target location as cancerous, non-cancerous, as a specific type of cancer, among other things. If the at least one criterion applied by preliminary screening test determines that the target location is not suitable for proceeding with illumination and response measurement for actually performing the tissue characterization or classification, the user can be directed, e.g., via a user interface, to take some action (e.g., to clean the target location, to dry the target location, to change an applied pressure of a probe or transducer pressed against the tissue specimen 102 at the target location, to move the probe or transducer to a different target location, or the like).

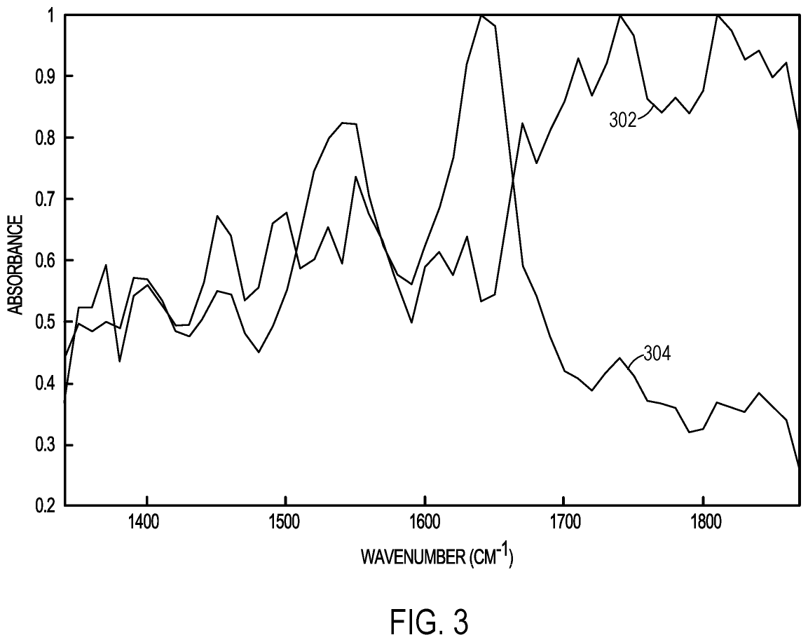

FIG. 3 is a spectral response graph of light absorbance vs. wavenumber. The trace 302 is an illustrative example of a spectral response at a target location that has a layer of dry or dead skin, which could confound accurate tissue characterization or classification at that target location. The trace 304 is an illustrative example of a spectral response at the same target location after cleaning to remove the layer of dry or dead skin. The large peaks that appear at around 1550 cm−1 and 1650 cm−1 of the trace 304 (but not in the dry skin trace 302) are useful for the actual tissue characterization or classification. Therefore, a preliminary screening test that can identify suitability of a target location for performing the actual tissue characterization or classification can be configured to distinguish between the spectral response shown by the trace 302 and the spectral response shown by the trace 304. This can include comparing a spectral response of the pre-screening to one or more tissue similarity criteria, such as to a template, to one or more threshold conditions, or the like to determine whether there are peaks present at or near each of 1550 cm−1 and 1650 cm−1, relative to a baseline. If such peaks are present at or near around 1550 cm−1 and 1650 cm-1, this can be used as an indication that the target location can be deemed suitable for performing the actual tissue characterization or classification. If such peaks are absent at or near around 1550 cm−1 and 1650 cm−1, this can be used as an indication that the target location can be deemed unsuitable for performing the actual tissue characterization or classification, in which case cleaning the site or some other user action may be instructed.

At 207 of FIG. 2, performing the preliminary screening test can include determining a similarity of one or more portions of a mid-range infrared response spectrum to that of “characterizable” tissue of interest, and indicating the suitability of the target location based at least in part on the determined similarity at the target location to tissue meeting or exceeding at least one tissue similarity criterion. Conversely, the preliminary screening test can include determining a similarity of one or more portions of a mid-range infrared response spectrum to that of “non-characterizable” tissue of interest or tissue not of interest (e.g., dry skin, dead skin, keratinous skin, or the like), in which case non-suitability of the target location can be indicated based at least in part on the determined similarity at the target location to that of “non-characterizable” tissue of interest or tissue not of interest. Either or both of such preliminary screening tests can be performed at 207 to help determine suitability of the target location.

At 207 of FIG. 2, performing the preliminary screening test to determine the suitability or the target location can include determining a similarity of one or more portions of the mid-range infrared response spectrum to that of one or more of sunscreen, skin lotion, other cosmetic, rubbing alcohol, soap residue, water, saline, or other cleaning agent, or cotton residue or fabric residue or other contaminant, and indicating the suitability based at least in part on the determined similarity to the contaminant not meeting at least one contaminant criterion.

At 207 of FIG. 2, performing the preliminary screening test to determine the suitability or the target location can include determining a similarity of one or more portions of the mid-range infrared response spectrum to that of tissue without sufficient instrument contact at the tissue location, and indicating the suitability based at least in part on the determined similarity to the tissue without sufficient instrument contact not meeting at least one tissue contact criterion.

### C. Selecting Between Specific Tissue Characterization Models Using Information From Preliminary Screening Test or Other Information

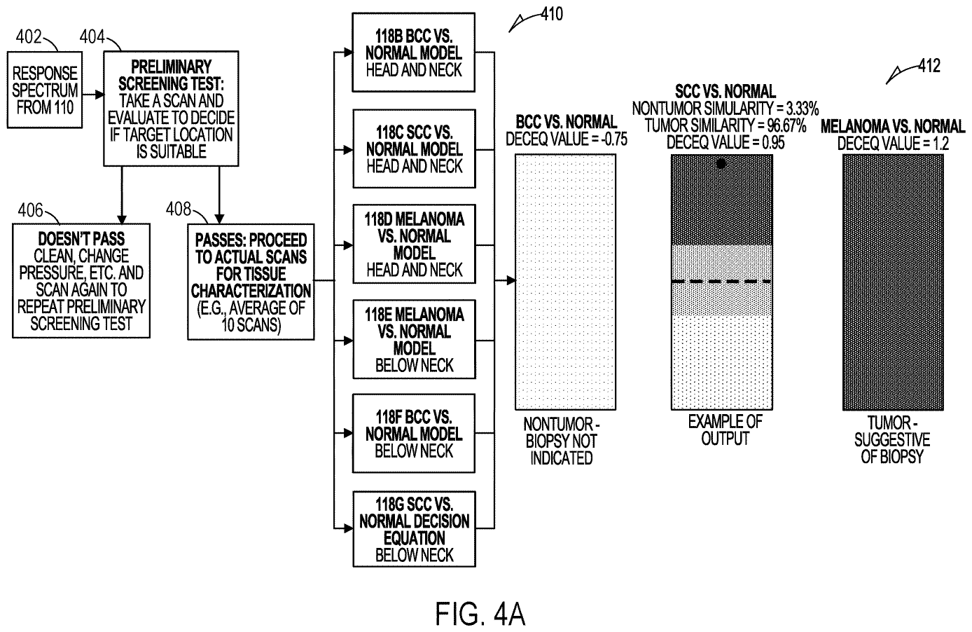

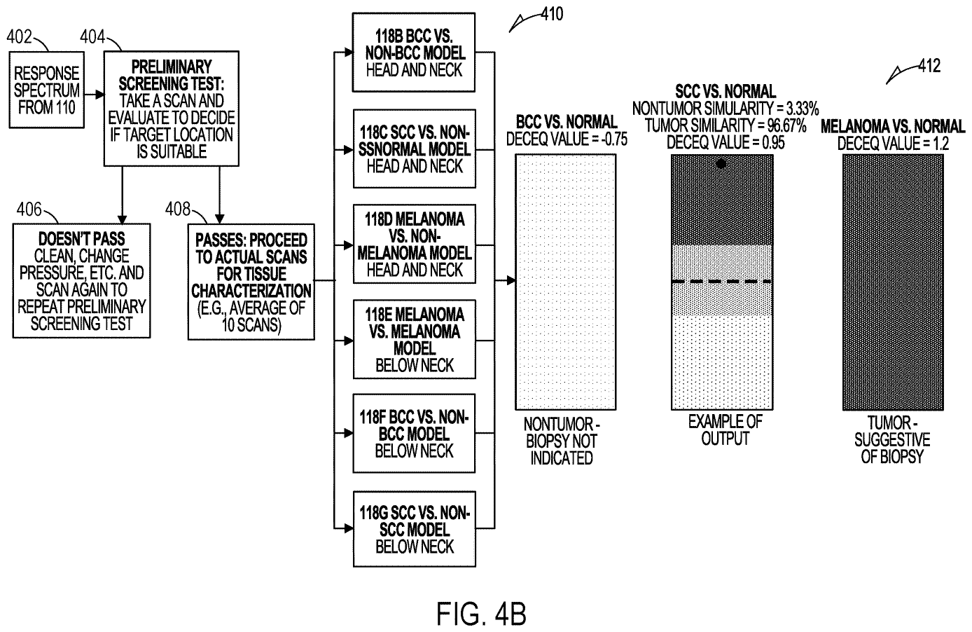

FIG. 4A is a block diagram illustrating an approach of: (1) using the preliminary screening test before proceeding to actual tissue characterization or classification; (2) selecting between different specific trained learning models 118, such as based on information from the preliminary screening test, user input information, or other information; and (3) presenting information to a user about the tissue characterization or classification, such as which can convey a binary tissue characterization or classification and a degree of confidence or similarity to one or both classes in the binary tissue characterization or classification.

At 402, a response spectrum can be obtained, such as by the electromagnetic energy response detector 110 in response to illumination by the electromagnetic energy illuminator 108. This can occur in response to a Power-On/Reset (POR) or other bootup or other initial or other triggering event, and the response spectrum can be used to perform a system diagnostic test. Among other things, the system diagnostic test can check whether the readout display is working, check whether an optical fiber or fiber bundle is functioning and unbroken, check lens or crystal alignment to the fiber or fiber bundle, check whether the light source is operational, or additionally or alternatively perform one or more other diagnostics, which can be based at least in part on the response spectrum, if desired.

At 404, a preliminary screening test can be performed, such as to determine suitability of the target location associated with the tissue specimen 102, such as described herein with respect to 207 of FIG. 2.

At 406, if the preliminary screening test at 404 is not passed, indicating that the target location is not suitable for tissue characterization or classification, then the target location can be changed, the probe or transducer pressure against the target location can be adjusted, or the like, and process flow can return to 404 to repeat the preliminary screening test at 404.

At 408, if the preliminary screening test at 404 is passed, indicating that the target location is suitable for tissue characterization or classification, then actual tissue characterization or classification can commence. This can include the controller 106 directing the electromagnetic energy illuminator 108 to illuminate the tissue specimen 102 at the target location, such as by performing a “scan” at various illumination wavenumbers of interest to tissue characterization. Similarly, the controller 106 can direct the electromagnetic energy response detector 110 to detect the corresponding response spectrum at 402 for the various illumination wavenumbers included in the scan.

At 408, multiple scans can be performed, and an average or other central tendency of the corresponding response spectra can be determined. This average or other central tendency of the corresponding response spectra can then be used for selecting and/or applying a specific trained learning model 118A . . . 118N for performing the tissue characterization or classification.

At 410, the specific trained learning model can be selected, applied, or both based on one or more factors, such as can include information particular to the target location, which may be obtained from the user (e.g., via a user interface), from the preliminary screening test, from a patient's Electronic Medical Record (EMR), or from another source. The different available trained models 118A . . . 118N can be trained, selected between, and applied specific to one or more of: (1) location (e.g., head and neck, oral cavity, below neck, external skin, subdermal tissue, breast tissue, etc.); specific cancer (e.g., squamous cell carcinoma (SCC), basal cell carcinoma (BCC), melanoma, etc.); specific racial or genealogy characteristic; or a combination of these, among others. To the extent that advance knowledge of one or more attributes is available and can be used to help select, apply, or both, at 410, one or more trained models that have been trained specifically on other tissue samples having that same attribute. Using the more specific trained model can help improve one or both of the sensitivity or specificity of the tissue characterization or classification.

At 410, one or more specific trained learning models 118 can be selected and applied, such as based on information from the preliminary screening test at 404, information obtained via the user interface or display 122, information based on the patient's EMR, or the like. In a variation on this approach, multiple ones of the specific trained learning models 118 can be applied in parallel to the central tendency of the corresponding response spectra, and the respective characterizations obtained using the different specific trained learning models 118 being applied in parallel can be combined, such as by weighting or another approach, such as explained further below.

At 412, an output of the tissue characterization or classification obtained by applying one or more of the specific trained models 118 to the central tendency of the response spectrum can be evaluated and displayed, such as on the user interface or display 122. This can include a binary tissue characterization or classification result, such as one of “Nontumor-Biopsy Not Indicated” (e.g., which can be indicated by a displayed green or other characteristic indicator) or “Tumor Suggestive of Biopsy” (e.g., which can be indicated by a displayed red or other characteristic indicator). This can also include using one or both of the controller 106 or the signal processor 114 for algorithmically or, using a trained model, determining at least one of a similarity or dissimilarity metric. Based on the determined metric, the user interface or display 122 can display an intermediate indicator, such as which can indicate a degree to which the tissue characterization or classification falls into one of the two binary tissue characterization or classifications. For example this can be indicated by an indicator marker on a colored indicator that transitions between green and red to indicate the opposite binary classes, with the location of the indicator marker on the colored indicator indicates a degree to which the tissue characterization or classification is indicative of one of the two binary classes.

FIG. 4B is a block diagram, similar to FIG. 4A, but in which at 410 the model 118B can include BCC vs. non-BCC model for the head and neck, instead of BCC vs. Normal as described with respect to FIG. 4A. Thus, in FIG. 4B, non-BCC can be trained using data from Normal, Melanoma, and SCC patients and not just limited to being trained using “Normal” patients against BCC patients, as was the case with FIG. 4A. Similarly, in FIG. 4B, the model 118C can include SCC vs. non-SCC for the head and heck, the model 118D can include Melanoma vs. Non-Melanoma for the head and neck, the model 118E can include Melanoma vs. Non-Melanoma for below the neck, the model 118F can include BCC vs. non-BCC for below the neck, and the model 118G can include SCC vs. non-SCC for below the neck. Any of the models at 410 in FIG. 4B can be used or made available in combination with any of the models at 410 in FIG. 4A.

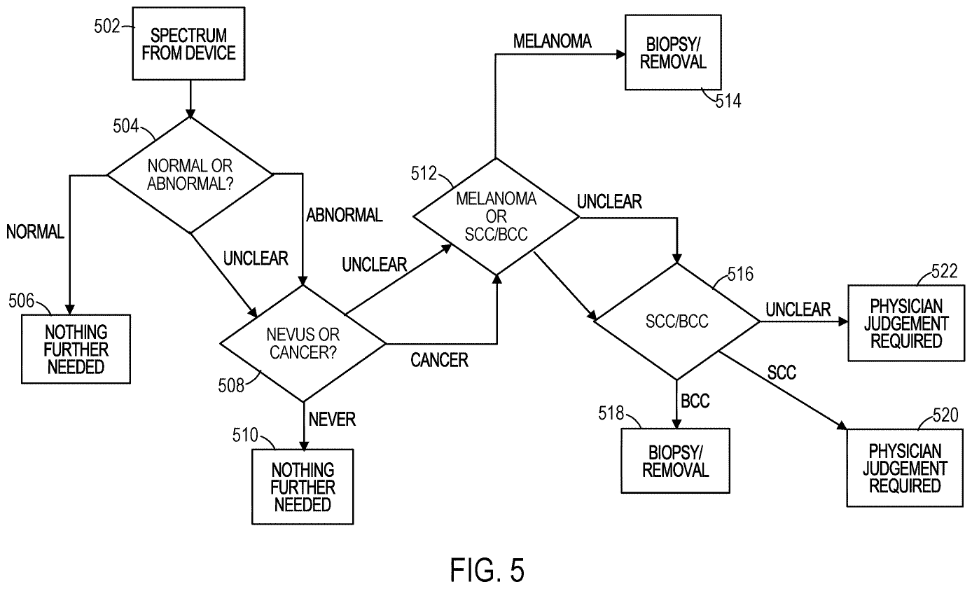

FIG. 5 is a flow chart illustrating an example of combining different tissue characterization or classification results obtained from corresponding different trained specific learning models 118, such as mentioned above. Combining different tissue characterization or classification results obtained from corresponding different trained specific learning models 118 can include appropriately weighting these different tissue characterization or classification results. Such weighting can be performed using a convolutional neural network (CNN), or manually creating such weights such as by multiplying SVM decision equation values by a certain number. Such weighting can also be determined using Principal Component Analysis (PCA) or Single Value Decomposition (SVD), or using information from the preliminary screening test.

FIG. 5 illustrates an example of an evaluation or decision sequence that can be used to proceed towards selecting and applying various specific trained learning models 118 for tissue characterization or classification.

At 502, a response spectrum can be received from the electromagnetic energy response detector 110, such as in response to illuminations at different specified wavenumbers by the electromagnetic energy illuminator 108, both of which can be controlled using the controller 106.

At 504, the classifier 120 of the computing device 104 can be used to perform tissue characterization or classification using the response spectrum received at 502, such as after optionally performing a preliminary screening test to determine suitability of the target location at which the tissue specimen 102 is located. The tissue characterization or classification at 504 can use a first trained learning model 118A, such as to characterize or classify the tissue specimen 102 as one of normal or abnormal, such as using a learning model 118 that has been trained for that particular type of tissue characterization or classification. This tissue characterization or classification can include an indication of a degree of confidence or a degree of similarity associated with a binary tissue characterization or classification, if desired, such as explained above. If the tissue characterization or classification at 504 is deemed “normal,” then nothing further is needed, as indicated at 506. If the tissue characterization or classification at 504 is deemed “abnormal” or, based on the degree of confidence or similarity, “unclear,” then process flow can proceed to 508.

At 508, the classifier 120 of the computing device 104 can be used to perform tissue characterization or classification using the response spectrum received at 502. The tissue characterization or classification at 508 can use a trained learning model 118B (e.g., a different trained learning model 118 than what was employed at 504), such as to characterize or classify the tissue specimen 102 as one of nevus (e.g., a birthmark or mole) or cancer, such as using a learning model 118B that has been trained for that particular type of tissue characterization or classification. This tissue characterization or classification can include an indication of a degree of confidence or a degree of similarity associated with a binary tissue characterization or classification, if desired, such as explained above. If the tissue characterization or classification at 508 is deemed “nevus,” then nothing further is needed, as indicated at 510. If the tissue characterization or classification at 508 is deemed “cancer” or, based on the degree of confidence or similarity, is deemed “unclear,” then process flow can proceed to 512.

At 512, the classifier 120 of the computing device 104 can be used to perform tissue characterization or classification using the response spectrum received at 502. The tissue characterization or classification at 512 can use a trained learning model 118C (e.g., a different trained learning model 118 than what was employed at 504 or 508), such as to characterize or classify the tissue specimen 102 as one of (1) melanoma or (2) SCC/BCC, such as using a learning model 118C that has been trained for that particular type of tissue characterization or classification. This tissue characterization or classification can include an indication of a degree of confidence or a degree of similarity associated with a binary tissue characterization or classification, if desired, such as explained above. If the tissue characterization or classification at 512 is deemed “melanoma,” then at 514 an indication can be presented to the user recommending biopsy or removal of tissue at the target location of the tissue specimen 102. If the tissue characterization or classification at 512 is deemed to be one of SCC/BCC or, based on the degree of confidence or similarity, “unclear,” then process flow can proceed to 516.

At 516, the classifier 120 of the computing device 104 can be used to perform tissue characterization or classification using the response spectrum received at 502. The tissue characterization or classification at 516 can use a trained learning model 118D (e.g., a different trained learning model 118 than what was employed at 504 or 508 or 512), such as to characterize or classify the tissue specimen 102 as one of (1) SCC or (2) BCC, such as using a learning model 118D that has been trained for that particular type of tissue characterization or classification. This tissue characterization or classification can include an indication of a degree of confidence or a degree of similarity associated with a binary tissue characterization or classification, if desired, such as explained above. If the tissue characterization or classification at 516 is deemed “BCC,” then at 518 an indication can be presented to the user recommending biopsy or removal of tissue at the target location of the tissue specimen 102. If the tissue characterization or classification at 516 is deemed to be SCC then at 520 an indication (e.g., a different indication than that presented at 518) can be presented to the user recommending biopsy or removal of tissue at the target location of the tissue specimen 102. If the tissue characterization or classification at 516 is deemed to be “unclear” then at 522 an indication can be presented to the user requesting physician judgment for further evaluation, characterization, or classification of the tissue specimen 102 at the target location.

Although FIG. 5 has been described emphasizing a sequential approach of applying multiple models 504, 508, 512, and 516 to the target illumination response spectrum 502 from the device, alternatively, these multiple models 504, 508, 512, and 516 can be applied concurrently in parallel to the target illumination response spectrum 502 from the device, if desired, or a combination of sequential and concurrent model application can be used.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described.

However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. For example, a numbered list of Aspects is given below.

Aspect 1 can include or use, or a system, device, product, computer-assisted method, or device-readable storage medium that can include or use, or include instructions for performing acts that can include or an electromagnetic energy illumination response signal from a tissue sample for mid-range infrared spectroscopic analysis and tissue characterization. This can include performing a preliminary screening test at a target location of interest of a patient. The screening test can include performing a mid-range infrared pre-characterization illumination and spectroscopic response analysis at the target location. This can also include configuring or using a computing device to determine a suitability of the target location for using a trained learning model for performing spectroscopic characterization at the target location. Aspect 1 can also include or use, in response to the screening test meeting at least one criterion indicating the suitability, then performing a mid-range infrared tissue characterization illumination and spectroscopic response analysis at the target location using the computing device to access a plurality of different trained learning models stored in a memory in or coupled to the computing device. Aspect 1 can also include or use the computing device for generating an indication for presentation to a user for indicating characterization of tissue at the tissue location.

Aspect 2 can include, by itself or in combination with one or more portions of Aspect 1, in response to the screening test meeting at least one criterion indicating the suitability, then performing a mid-range infrared tissue characterization illumination and spectroscopic response analysis at the target location using the computing device to access and select between a plurality of different trained learning models stored in a memory in or coupled to the computing device, the plurality of different trained learning models selected between based on a result determined using the preliminary screening test.

Aspect 3 can include, by itself or in combination with one or more portions of any one or more of Aspects 1-2, in response to the screening test meeting at least one criterion indicating the suitability, then performing a mid-range infrared tissue characterization illumination and spectroscopic response analysis at the target location using the computing device to access and select between a plurality of different trained learning models stored in a memory in or coupled to the computing device, the plurality of different trained learning models selected between based on input anatomical information about the target location.

Aspect 4 can include, by itself or in combination with one or more portions of any one or more of Aspects 1-3, in response to the screening test meeting at least one criterion indicating the suitability, then performing a mid-range infrared tissue characterization illumination and spectroscopic response analysis at the target location using the computing device to access and select between a plurality of different trained learning models stored in a memory in or coupled to the computing device, the plurality of different trained learning models selected between based on a result of one or more applied individual ones of the different trained learning models.

Aspect 5 can include, by itself or in combination with one or more portions of any one or more of Aspects 1-4, wherein the performing the preliminary screening test comprises determining a similarity of one or more portions of a mid-range infrared response spectrum to that of tissue, and indicating the suitability based at least in part on the determined similarity to tissue meeting or exceeding at least one tissue similarity criterion.

Aspect 6 can include, by itself or in combination with one or more portions of any one or more of Aspects 1-5, wherein performing the preliminary screening test comprises determining a similarity of one or more portions of the mid-range infrared response spectrum to that of one or more of sunscreen, skin lotion, other cosmetic, rubbing alcohol, soap residue, water, or other cleaning agent, or other contaminant, and indicating the suitability based at least in part on the determined similarity to the contaminant not meeting at least one contaminant criterion.

Aspect 7 can include, by itself or in combination with one or more portions of any one or more of Aspects 1-6, wherein the performing the preliminary screening test comprises determining a similarity of one or more portions of the mid-range infrared response spectrum to that of tissue without sufficient instrument contact at the tissue location, and indicating the suitability based at least in part on the determined similarity to the tissue without sufficient instrument contact not meeting at least one tissue contact criterion.

Aspect 8 can include, by itself or in combination with one or more portions of any one or more of Aspects 1-7, wherein performing a mid-range infrared diagnostic illumination and spectroscopic response analysis at the target location includes using a central tendency response spectrum from multiple illumination and response acquisition scans, to which are applied one or more of the plurality of different trained learning models stored in a memory in or coupled to the computing device, the plurality of different trained learning models including at least a first trained learning model trained for discriminating between normal tissue and a first specific type of cancer and a second trained learning model trained for discriminating between normal tissue a different second specific type of cancer.

Aspect 9 can include, by itself or in combination with one or more portions of any one or more of Aspects 1-8, wherein the performing a mid-range infrared diagnostic illumination and spectroscopic response analysis at the target location includes using the computing device to access and apply a plurality of different trained learning models stored in a memory in or coupled to the computing device, wherein the plurality of different trained learning models includes at least two of: (1) a first trained learning model trained for discriminating between normal tissue and a first specific type of cancer being melanoma; (2) a second trained learning model trained for discriminating between normal tissue a different second specific type of cancer being squamous cell carcinoma (SCC); and (3) a third trained learning model trained for discriminating between normal tissue and a different third specific type of cancer being basal cell carcinoma (BCC).

Aspect 10 can include, by itself or in combination with one or more portions of any one or more of Aspects 1-9, wherein the performing a mid-range infrared diagnostic illumination and spectroscopic response analysis at the target location includes using the computing device to access and apply a plurality of different trained learning models stored in a memory in or coupled to the computing device, includes the plurality of different trained learning models including different associated body locations of the target location.

Aspect 11 can include, by itself or in combination with one or more portions of any one or more of Aspects 1-10, wherein the generating the indication for presentation to a user for indicating characterization of tissue at the tissue location includes the indication being capable of indicating: (1) a non-tumor characterization; (2) a specific type of cancer characterization; and (3) an intermediate indication quantifying a degree of similarity to at least one of (1) or (2).

Aspect 12 can include, by itself or in combination with one or more portions of any one or more of Aspects 1-11, wherein the performing the preliminary screening test includes at least one of: a baseline comparison to a baseline of one of a tissue response spectrum or a non-tissue response spectrum; a noise comparison to a noise of one of a tissue response spectrum or a non-tissue response spectrum; a metric of amplitude peaks comparison to a metric of amplitude peaks of one of a tissue response spectrum or a non-tissue response spectrum; or a dot product comparison with one or more spectra based on a corresponding sample that includes one or more specified molecules.

Aspect 13 can include, by itself or in combination with one or more portions of any one or more of Aspects 1-12, wherein the performing the preliminary screening test includes a metric of amplitude peaks comparison to a metric of amplitude peaks of one of a tissue response spectrum or a non-tissue response spectrum, wherein the metric includes at least one of: an amplitude ratio between two specified amplitude peaks; or a wavenumber location of one or more specified amplitude peaks.

Aspect 14 can include, by itself or in combination with one or more portions of any one or more of Aspects 1-13, further comprising individually weighting at least two of the trained learning models in the plurality of different trained learning models.

Aspect 15 can include, by itself or in combination with one or more portions of any one or more of Aspects 1-14, further comprising using the weighting for selecting between individual ones of the trained learning models in the plurality of different trained learning models.

Aspect 16 can include, by itself or in combination with one or more portions of any one or more of Aspects 1-15, wherein the weighting includes determining weights using at least one of Principal Component Analysis (PCA) or Single Value Decomposition (SVD).

Aspect 17 can include, by itself or in combination with one or more portions of any one or more of Aspects 1-16, including or using a system including one of processor circuitry or a device-readable medium for using an electromagnetic energy illumination response signal from a tissue sample for spectroscopic analysis and tissue classification, including or programmed with stored instructions that, when executed by processor circuitry, perform acts of: performing a preliminary screening test at a target location of interest of a patient, the screening test including a mid-range infrared pre-characterization illumination and spectroscopic response analysis at the target location using a computing device to determine a suitability of the target location for using a trained learning model for performing spectroscopic characterization at the target location; in response to the screening test meeting at least one criterion indicating the suitability, then performing a mid-range infrared tissue characterization illumination and spectroscopic response analysis at the target location using the computing device to access a plurality of different trained learning models stored in a memory in or coupled to the computing device; and using the computing device, generating an indication for presentation to a user for indicating characterization of tissue at the tissue location.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like.

Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A computer-assisted method of using an electromagnetic energy illumination response signal from a tissue sample for mid-range infrared spectroscopic analysis and tissue characterization, the computer-assisted method comprising acts of:
performing a preliminary screening test at a target location of interest of a patient, the screening test including a mid-range infrared pre-characterization illumination and spectroscopic response analysis at the target location using a computing device to determine a suitability of the target location for using a trained learning model for performing spectroscopic characterization at the target location, wherein the performing the preliminary screening test includes a metric of a smaller-numbered set of amplitude peaks comparison to a metric of amplitude peaks of a living tissue response spectrum, wherein the metric includes at least one of:
an amplitude ratio between two specified amplitude peaks; or
a wavenumber location of one or more specified amplitude peaks;
only in response to the screening test meeting at least one criterion indicating the suitability as being representative of living tissue being present at the target location, then performing a mid-range infrared tissue characterization illumination and spectroscopic response analysis of a larger-numbered set of amplitude peaks at the target location using the computing device to access a different trained learning model stored in a memory in or coupled to the computing device to discriminate between non-tumor and cancer at the target location; and
using the computing device, generating an indication for presentation to a user for indicating characterization of tissue at the tissue location based on mid-range infrared tissue characterization illumination and spectroscopic response analysis of the larger-numbered set of amplitude peaks at the target location, the indication being capable of indicating at least: (1) a non-tumor characterization; (2) a cancer characterization.

2. The computer-assisted method of claim 1, comprising:
in response to the screening test meeting at least one criterion indicating the suitability, then performing a mid-range infrared tissue characterization illumination and spectroscopic response analysis at the target location using the computing device to access and select between a plurality of different trained learning models stored in a memory in or coupled to the computing device, the plurality of different trained learning models selected between based on a result determined using the preliminary screening test.

3. The computer-assisted method of claim 1, comprising:
in response to the screening test meeting at least one criterion indicating the suitability, then performing a mid-range infrared tissue characterization illumination and spectroscopic response analysis at the target location using the computing device to access and select between a plurality of different trained learning models stored in a memory in or coupled to the computing device, the plurality of different trained learning models selected between based on input anatomical information about the target location.

4. The computer-assisted method of claim 1, comprising:
in response to the screening test meeting at least one criterion indicating the suitability, then performing a mid-range infrared tissue characterization illumination and spectroscopic response analysis at the target location using the computing device to access and select between a plurality of different trained learning models stored in a memory in or coupled to the computing device, the plurality of different trained learning models selected between based on a result of one or more applied individual ones of the different trained learning models.

5. The computer-assisted method of claim 1, wherein the performing the preliminary screening test comprises determining a similarity of one or more portions of a mid-Filing range infrared response spectrum to that of tissue, and indicating the suitability based at least in part on the determined similarity to tissue meeting or exceeding at least one tissue similarity criterion.

6. The computer-assisted method of claim 1, wherein performing the preliminary screening test comprises determining a similarity of one or more portions of the mid-range infrared response spectrum to that of one or more of sunscreen, skin lotion, other cosmetic, rubbing alcohol, soap residue, water, or other cleaning agent, or other contaminant, and indicating the suitability based at least in part on the determined similarity to the contaminant not meeting at least one contaminant criterion.

7. The computer-assisted method of claim 1, wherein the performing the preliminary screening test comprises determining a similarity of one or more portions of the mid-range infrared response spectrum to that of tissue without sufficient instrument contact at the tissue location, and indicating the suitability based at least in part on the determined similarity to the tissue without sufficient instrument contact not meeting at least one tissue contact criterion.

8. The computer-assisted method of claim 1, wherein performing a mid-range infrared diagnostic illumination and spectroscopic response analysis at the target location includes using a central tendency response spectrum from multiple illumination and response acquisition scans, to which are applied one or more of the plurality of different trained learning models stored in a memory in or coupled to the computing device, the plurality of different trained learning models including at least a first trained learning model trained for discriminating between normal tissue and a first specific type of cancer and a second trained learning model trained for discriminating between normal tissue a different second specific type of cancer.

9. The computer-assisted method of claim 1, wherein the performing a mid-range infrared diagnostic illumination and spectroscopic response analysis at the target location includes using the computing device to access and apply a plurality of different trained learning models stored in a memory in or coupled to the computing device, wherein the plurality of different trained learning models includes at least two of:
a first trained learning model trained for discriminating between normal tissue and a first specific type of cancer being melanoma;
a second trained learning model trained for discriminating between normal tissue a different second specific type of cancer being squamous cell carcinoma (SCC); and
a third trained learning model trained for discriminating between normal tissue and a different third specific type of cancer being basal cell carcinoma (BCC).

10. The computer-assisted method of claim 1, wherein the performing a mid-range infrared diagnostic illumination and spectroscopic response analysis at the target location includes using the computing device to access and apply a plurality of different trained learning models stored in a memory in or coupled to the computing device, includes the plurality of different trained learning models including different associated body locations of the target location.

11. The computer-assisted method of claim 1, wherein the generating the indication for presentation to a user for indicating characterization of tissue at the tissue location includes the indication being capable of indicating: (1) a non-tumor characterization; (2) a specific type of cancer characterization; and (3) an intermediate indication quantifying a degree of similarity to at least one of (1) or (2).

12. The computer-assisted method of claim 1, wherein the performing the preliminary screening test includes at least one of:
a baseline comparison to a baseline of one of a tissue response spectrum or a non-tissue response spectrum;
a noise comparison to a noise of one of a tissue response spectrum or a non-tissue response spectrum;
a metric of amplitude peaks comparison to a metric of amplitude peaks of one of a tissue response spectrum or a non-tissue response spectrum; or
a dot product comparison with one or more spectra based on a corresponding sample that includes one or more specified molecules.

13. (canceled) The computer-assisted method of claim 12, wherein the performing the preliminary screening test includes a metric of amplitude peaks comparison to a metric of amplitude peaks of one of a tissue response spectrum or a non-tissue response spectrum, wherein the metric includes at least one of:
an amplitude ratio between two specified amplitude peaks; or a wavenumber location of one or more specified amplitude peaks.

14. The computer-assisted method of claim 1, further comprising individually weighting at least two trained learning models in a plurality of different trained learning models.

15. The computer-assisted method of claim 14, further comprising using the weighting for selecting between individual ones of the trained learning models in the plurality of different trained learning models.

16. The computer-assisted method of claim 14, wherein the weighting includes determining weights using at least one of Principal Component Analysis (PCA) or Single Value Decomposition (SVD).

17. A system including one of processor circuitry or a device-readable medium for using an electromagnetic energy illumination response signal from a tissue sample for spectroscopic analysis and tissue classification, including or programmed with stored instructions that, when executed by processor circuitry, perform acts of:
performing a preliminary screening test at a target location of interest of a patient, the screening test including a mid-range infrared pre-characterization illumination and spectroscopic response analysis at the target location using a computing device to determine a suitability of the target location for using a trained learning model for performing spectroscopic characterization at the target location, wherein the performing the preliminary screening test includes a metric of a smaller-numbered set of amplitude peaks comparison to a metric of amplitude peaks of a living tissue response spectrum, wherein the metric includes at least one of:
an amplitude ratio between two specified amplitude peaks: or
a wavenumber location of one or more specified amplitude peaks;
only in response to the screening test meeting at least one criterion indicating the suitability as being representative of living tissue at the target location, then performing a mid-range infrared tissue characterization illumination and spectroscopic response analysis of a larger-numbered set of amplitude peaks at the target location using the computing device to access a of different trained learning model stored in a memory in or coupled to the computing device to discriminate between non-tumor and cancer at the target location, and
using the computing device, generating an indication for presentation to a user for indicating characterization of tissue at the tissue location based on mid-range infrared tissue characterization illumination and spectroscopic response analysis of the larger-numbered set of amplitude peaks at the target location, the indication being capable of indicating at least: (1) a non-tumor characterization: (2) a cancer characterization.
